(19) United States
(12) Patent Application Publication
(10) Pub. No.: US 2008/0161413 A1
(43) Pub. Date: Jul. 3, 2008

(54) Agent for increasing adiponectin in blood

(75) Inventor: Yumika OKADA, Shizuoka (JP)

Correspondence Address: McLELAND PATENT LAW OFFICE, P.L.L.C., 11320 RANDOM HILLS ROAD, SUITE 250, FAIRFAX, VA, 22030, US

(73) Assignee: YAMAHA HATSUDOKI KABUSHIKI KAISHA, Iwata-shi (JP)

(21) Appl. No.: 11/963,195

(22) Filed: Dec. 21, 2007

(30) Foreign Application Priority Data
Dec. 27, 2006 (JP) 2006-352373

(51) Int. Cl. A61K 31/122; A61P 7/00

(52) U.S. Cl. 514/691

(57) ABSTRACT

Novel, safe, and highly effective agents for increasing adiponectin in blood that contain astaxanthin and/or an ester thereof as an active component are provided. Due to an increase in the amount of adiponectin in blood, the action of adipocytokine such as TNFα can be decreased, or the action of biologically active substances such as insulin can be protected from the adverse action of adipocytokine. It is expected that, as a result, insulin resistance and hyperinsulinemia caused by adipocytokine, and metabolic syndrome are prevented or suppressed. Furthermore, it is expected that progress of arteriosclerosis, which can lead to cardiovascular diseases and cerebrovascular diseases, is inhibited by ingestion of the agent for increasing adiponectin in blood of the present invention, because arteriosclerosis progresses due to a decrease in adiponectin.

### BACKGROUND OF THE INVENTION

1. Field of the Invention

The present invention relates to an agent for increasing adiponectin in blood and a method for increasing adiponectin in blood.

2. Description of the Related Art

Life-style related diseases such as hypertension, hyperlipemia, and diabetes tend to occur concurrently. It has become clear that this is attributable to obesity (visceral fat obesity) in which fat accumulates around the internal organs. A pathological condition induced by visceral fat obesity and characterized by the coincidence of many risk factors causing arteriosclerosis is referred to as metabolic syndrome, and metabolic syndrome is a risk factor for arteriosclerosis as well as cardiovascular and cerebrovascular diseases caused thereby.

Obesity is caused when caloric expenditure is lower than caloric intake and the energy source that thus has not been expended accumulates as body fat. The causes of body fat accumulation due to excess energy include lack of exercise, improper eating habit, stress, lipid metabolism abnormality (disorder), excessive secretion of insulin, enlargement of adipocytes, and lack of brown adipocytes. Visceral fat described above is fat found in the mesentery located within the peritoneal cavity, and visceral adipocytes tend to store fat within the individual cells.

When fat accumulates in adipocytes, various types of adipokines (e.g., TNFα, resistin, etc.) are secreted by the adipocytes, causing insulin resistance (i.e., decreased insulin sensitivity). As a result, blood glucose levels can no longer be sufficiently lowered, so that insulin is over-secreted in order to control blood glucose levels, resulting in hyperinsulinemia. When hyperinsulinemia occurs, metabolic syndrome is caused by the action of excess insulin on lipid metabolism and the like.

Adiponectin is a beneficial adipokine. Adiponectin activates AMP-activated protein kinase (AMPK) in the skeletal muscle and the liver, thereby promoting fatty acid burning and glucose uptake, and alleviating insulin resistance (Takashi Kadowaki et al., “The Role of Adiponectin in Molecular Mechanisms of Diabetes and Cardiovascular Diseases”, proceedings of The 128th Japanese Association of Medical Sciences Symposium on “Diabetes Mellitus and Atherosclerosis”, Dec. 2, 2004, pp. 34-45). Differentiation of preadipocytes into adipocytes is accompanied by induction of expression of adiponectin. When adipocytes are not hypertrophied, adiponectin is actively secreted from adipocytes. On the other hand, when adipocytes are hypertrophied, the action of adiponectin is decreased by the above-described TNFα and the other adipokines. Furthermore, it has been reported that when adipocytes are hypertrophied, transcription of adiponectin is suppressed, and adiponectin becomes deficient, causing a metabolic abnormality, which leads to metabolic syndrome (Takashi Kadowaki et al., ibid). It has been reported also that a pathological condition of metabolic syndrome occurring due to lack of adiponectin is partially alleviated by supply of adiponectin (Takashi Kadowaki et al., ibid).

It is also known that adiponectin suppresses an increase in smooth muscle cells and suppresses adhesion of monocytes to endothelial cells (N. Ouchi et al., Circulation, 1999, vol. 100, no. 25, pp. 2473-2476). Furthermore, since inflammatory intimal thickening in the blood vessels increases in adiponectin-deficient mice, it seems that adiponectin has the action of suppressing arteriosclerosis. Thus, it seems that for preventing and treating metabolic syndrome, it is important to increase adiponectin in the body.

It has been described that a decrease in the production of adiponectin is in part attributable to oxidative stress caused by reactive oxygen species and the like (Japanese Laid-Open Patent Publication No. 2005-232059). It has been described that in particular, apocynin, which is an NADPH oxidase inhibitor, increases the production of adiponectin in obese/diabetic mice. NADPH oxidase is an enzyme having a relation to the production of reactive oxygen species, and expression thereof increases in adipocytes in the case of obesity. Based on these results, Japanese Laid-Open Patent Publication No. 2005-232059 has described that any compound having an antioxidative effect is useful against diseases that are caused by a low adiponectin level. However, it is not clear whether or not all of such antioxidants have a similar effect to that of apocynin.

Carotenoids are naturally-occurring substances having an antioxidative effect, and their various bioactivities have attracted interest. However, few studies have been conducted to investigate the action of carotenoids on obesity and adipocytes or insulin resistance. It has been reported only that a carotenoid derived from a vegetable or a fruit suppresses the differentiation induced by insulin of preadipocytes into adipocytes (Japanese Laid-Open Patent Publication No. 2003-95930). Furthermore, it has been reported that astaxanthin, which is a carotenoid, adjusts the concentration of neutral fat in blood (US 2006/0293387 A1). Japanese Laid-Open Patent Publication No. 2005-232059 has described carotenoids as an antioxidant, but any other relationship between the adiponectin and the carotenoids is not known at all.

### SUMMARY OF THE INVENTION

It is an object of the present invention to provide safe and highly effective agents for increasing adiponectin in blood.

The present invention provides an agent for increasing adiponectin in blood, comprising astaxanthin and/or an ester thereof as an active component.

The present invention also provides a method for increasing adiponectin in blood in a subject in need of an increase in adiponectin in blood, comprising administering to the subject an amount of astaxanthin and/or an ester thereof effective for increasing adiponectin in blood.

In an embodiment, the astaxanthin and/or the ester thereof is derived from a microalga belonging to the genus Haematococcus.

According to the present invention, novel and highly effective agents for increasing adiponectin in blood are provided. Ingestion of the agent for increasing adiponectin in blood of the present invention increases the amount of adiponectin in blood. Thus, it is expected that insulin resistance and the pathological condition of metabolic syndrome are prevented and alleviated. It is also expected that arteriosclerosis, which can lead to cardiovascular diseases and cerebrovascular diseases, is suppressed.

### BRIEF DESCRIPTION OF THE DRAWINGS

FIG. 1 is a graph showing the change over time in the average value of the concentration of adiponectin in blood of subjects before and after ingestion of astaxanthin capsules.

### DESCRIPTION OF THE PREFERRED EMBODIMENTS

Astaxanthin or an ester thereof contained in the agent for increasing adiponectin in blood of the present invention is a carotenoid represented by the following formula:

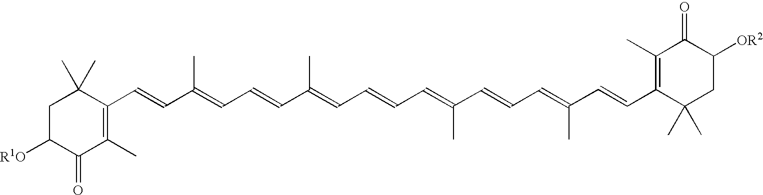

wherein R¹ and R² are both hydrogen in the case of astaxanthin, and R¹ and R² are each independently a hydrogen atom or a fatty acid residue provided that at least one of R¹ and R² is a fatty acid residue in the case of an ester of astaxanthin. Examples of the fatty acid residue in the ester of astaxanthin include, but are not limited to, saturated fatty acids such as palmitic acid and stearic acid or unsaturated fatty acids such as oleic acid, linoleic acid, α-linolenic acid, γ-linolenic acid, bishomo-γ-linolenic acid, arachidonic acid, eicosapentaenoic acid, and docosahexaenoic acid. The astaxanthin ester of the present invention can be any mono- or diester, homogeneous or non-homogeneous. Astaxanthin has a structure in which an additional oxo group and an additional hydroxy group are present at each end of a β-carotene molecule. On the other hand, in an ester form (e.g., as obtained in an extract from krill) in which the hydroxy groups at both ends are esterified with an unsaturated fatty acid and the like, the stability of the molecule is better.

Astaxanthin or an ester thereof used in the present invention may be chemically synthesized or derived from a naturally-occurring product. Examples of the naturally-occurring products in the latter case include red yeast; the shell of crustaceans such as Tigriopus (red water flea) and krills; and microalgae such as green algae, which contain astaxanthin and/or an ester thereof. In the present invention, any extract containing astaxanthin and/or esters thereof produced by any method can be used. Generally, extracts from those naturally-occurring products can be used, and the extracts may be crude or purified if necessary. In the present invention, a crude extract or a crushed powder of naturally-occurring products, or a purified product or a chemically synthesized product, if necessary, that contains such astaxanthin and/or esters thereof can be used either alone or in combination. In view of the chemical stability, an ester form of astaxanthin is preferably used.

The agent for increasing adiponectin in blood according to the present invention is effective in increasing the amount of adiponectin in blood. Thus, the action of adipocytokine such as TNFα can be decreased, or the action of metabolic activation substances such as insulin can be protected from the action of adipocytokine. As a result, insulin resistance can be prevented or suppressed. Therefore, hyperinsulinemia induced by insulin resistance, and subsequent metabolic syndrome can be prevented or suppressed.

In this specification, metabolic syndrome is diagnosed according to any of the methods accepted in this field. The diagnostic criteria for metabolic syndrome in Japan are that accumulation of visceral fat is present (waist circumference is 85 cm or more in men or 90 cm or more in women) and that at least two of the following conditions are present: hypertension including borderline hypertension, hyperlipemia or low HDL (high density lipoprotein), and hyperglycemia. Moreover, according to the U.S. guidelines for treatment of hyperlipemia (ATP III: Adult Treatment Panel III, NCEP National Cholesterol Education Program), metabolic syndrome is diagnosed when three of the following conditions (i) to (v) are present: (i) waist (abdominal circumference) is 102 cm or more in men or 88 cm or more in women; (ii) neutral fat in blood is 150 mg/dl or more; (iii) HDL cholesterol is less than 40 mg/dl in men or less than 50 mg/dl in women; (iv) blood pressure is such that the maximal blood pressure is 130 mmHg or more or the minimal blood pressure is 85 mmHg or more; and (v) fasting blood glucose level is 110 mg/dl or more. Furthermore, the diagnostic criteria for metabolic syndrome according to the WHO are that hyperinsulinemia is present or fasting blood glucose level is 110 mg/dl or more, plus that two of the following conditions (a) to (d) are present: (a) visceral obesity (waist/hip ratio>0.9 (men), >0.85 (women) or BMI of 30 or more or abdominal circumference of 94 cm or more); (b) lipid metabolism abnormality (neutral fat in blood of 150 mg/dl or more or HDL cholesterol of less than 35 mg/dl in men or less than 39 mg/dl in women); (c) hypertension (140/90 mmHg or more or during treatment with an antihypertensive agent); and (d) microalbuminuria (urinary albumin excretion rate of 20 μg/min or more or urinary albumin/creatinine ratio of 30 mg/g creatinine or more).

In this specification, prevention of metabolic syndrome refers to preventing or alleviating a state in which at least one of the conditions listed above as the diagnostic criteria is present. Moreover, the agent for increasing adiponectin in blood according to the present invention is useful also for prevention of a disease having a relation to insulin resistance. Examples of the disease having a relation to insulin resistance include various life-style related diseases, such as hyperlipemia, arteriosclerosis, hypertension, myocardial infarction, cerebrovascular disorders, cerebral infarction, angina pectoris, pancreatitis, diabetes, fatty liver, metabolic abnormalities, and obesity, and other diseases.

The route of administration of the agent for increasing adiponectin in blood according to the present invention may be either oral or parenteral. The dosage form is selected appropriately according to the route of administration. Examples thereof include parenteral solutions, infusion solutions, powders, granules, tablets, capsules, pills, enteric-coated preparations, troches, liquids for internal use, suspensions, emulsions, syrups, liquids for external use, poultices, nose drops, ear drops, eye drops, inhalants, ointments, lotions, suppositories, and enteral nutrients. These can be used either alone or in combination depending on the condition of a disease. To prepare these dosage forms, auxiliary substances commonly used in the field of pharmaceutical manufacturing technology, such as excipients, binders, antiseptics, antioxidants, disintegrators, lubricants, and flavoring agents, can be used as necessary.

The dose of the agent for increasing adiponectin in blood according to the present invention varies depending on the purpose of administration, the individual to be administered (sex, age, body weight, etc.), and the severity and nature of the disease, and can be determined by a person skilled in the art. Usually, the dose for an adult in terms of free or unesterified form of astaxanthin may be 0.1 mg to 2 g, preferably 4 mg to 500 mg per day in the case of oral administration, while it may be 0.01 mg to 1 g, preferably 0.1 mg to 500 mg per day in the case of parenteral administration.

The agent for increasing adiponectin in blood according to the present invention can be used not only as pharmaceuticals as described above, but also as the category of products regulated as “quasi-drugs”, cosmetics, food products, nutritional supplements, foods and drinks, and other similar products. When used as quasi-drugs or cosmetics, the agent may be used in conjunction with various auxiliary substances commonly used in the field of quasi-drugs or cosmetics, or other technologies, if necessary. Alternatively, when used as food products, nutritional supplements, or foods and drinks, the agent may be used in conjunction with additives commonly used for food products, for example, sweeteners, spices, seasonings, antiseptics, preservatives, germicides, and antioxidants, if necessary. The agent may be used in a desired form such as solution, suspension, syrup, granule, cream, paste, or jelly, or may be shaped, if necessary. The ratio of the agent contained in these products is not particularly limited, and can be selected appropriately according to the intended purpose, the mode of usage, and the amount of usage.

### EXAMPLES

### Preparation Example 1

### Preparation of Astaxanthin Capsule

Astaxanthin was prepared in the following manner. Haematococcus pluvialis K0084 strain was cultivated at 25° C. under irradiation with light while bubbling a gas containing 3% CO₂ into the medium and under nutrient stress condition (i.e. nitrogen source deprivation), and then was encysted. The encysted cells were disrupted by a bead beater, and a lipophilic fraction containing astaxanthin was extracted with ethanol. The extract was concentrated under reduced pressure, and the ethanol was evaporated to give an extract containing astaxanthin in an amount of 8.0% expressed in terms weight of the free form.

Soft capsules containing the components shown in Table 1 below per capsule were prepared using the extract containing astaxanthin in an amount of 8.0% expressed in terms weight of the free form.

TABLE 1

| Component | Weight |
|---|---|
| Haematococcus extract (Yamaha Hatsudoki K.K.) | 52 mg |
| Olive oil (The Nisshin OilliO Group, Ltd.) | 78 mg |
| Vitamin E (The Nisshin OilliO Group, Ltd.) | 20 mg |

The obtained soft capsules contained astaxanthin in an amount of 4 mg per capsule expressed in terms of weight of the free form.

### Example 1

### Effect on the Concentration of Adiponectin in Blood

Seventeen Japanese subjects suffering from mild metabolic syndrome between the ages of 20 to 65 ingested four of the astaxanthin capsules obtained in Preparation Example 1 per day (two capsules each time, immediately after the morning and evening meals) for 12 weeks. As described above, the diagnostic criteria for metabolic syndrome in Japan are that accumulation of visceral fat is present (waist circumference is 85 cm or more in men or 90 cm or more in women) and that at least two of the following conditions are present: hypertension including borderline hypertension, hyperlipemia or low HDL, and hyperglycemia.

Throughout the period in which the subjects were ingesting the astaxanthin capsules, they were prohibited to from initiating the new use of a drug or a health food product (including a supplement), and from starting a new diet/exercise therapy or a new physical therapy. Blood was collected from each subject before the morning meal on the day when the ingestion started, four weeks after the ingestion started, eight weeks after the ingestion started, and before the morning meal on the day following the day when the ingestion ended, to determine the concentration of adiponectin in blood. The concentration of adiponectin was measured using a human adiponectin ELISA kit (manufactured by Otsuka Pharmaceutical Co., Ltd.).

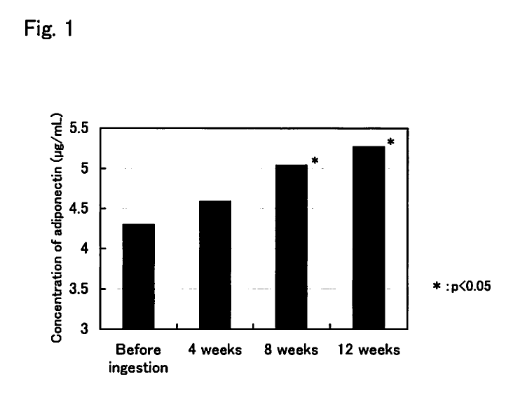

The results obtained by measuring the concentration of adiponectin in blood are shown in FIG. 1. FIG. 1 shows the change over time in the average value of the concentration of adiponectin in blood of all subjects before and after ingestion of the astaxanthin capsules. The values on the vertical axis are in μg/mL.

As can be seen from FIG. 1, it was confirmed that the concentration of adiponectin in blood was significantly increased in eight weeks and 12 weeks after the ingestion of astaxanthin started (t-test, p<0.05). Furthermore, in 12 weeks after the ingestion started, the amount of adiponectin was increased up to 123% of the amount before the ingestion. It is clearly shown that in this manner, the amount of adiponectin is gradually increased in human blood by continuous ingestion of astaxanthin.

The agent for increasing adiponectin in blood of the present invention is effective in increasing the amount of adiponectin in blood. Thus, the action of adipocytokine such as TNFα can be decreased, or the action of biologically active substances such as insulin can be protected from the adverse action of adipocytokine. As a result, insulin resistance and hyperinsulinemia caused by adipocytokine, and metabolic syndrome can be prevented or suppressed. Thus, it is expected that life-style related diseases such as hypertension, hyperlipemia, and diabetes are prevented or suppressed. Furthermore, it is expected that progress of arteriosclerosis, which can lead to cardiovascular diseases and cerebrovascular diseases, is inhibited, because arteriosclerosis progresses due to a decrease in adiponectin.

Astaxanthin and/or an ester thereof, which is an active component in the agent for increasing adiponectin in blood of the present invention, has been consumed in food for a long time and has very low toxicity; therefore, astaxanthin and/or an ester thereof has a very high degree of safety. Accordingly, this agent is preferably used not only as pharmaceuticals, but also as health food products and similar products used prophylactically on a daily basis.

The invention may be embodied in other forms without departing from the spirit or essential characteristics thereof. The embodiments disclosed in this application are to be considered in all respects as illustrative and not limiting. The scope of the invention is indicated by the appended claims rather than by the foregoing description, and all changes which come within the meaning and range of equivalency of the claims are intended to be embraced therein.

What is claimed is:

1. An agent for increasing adiponectin in blood, comprising astaxanthin and/or an ester thereof as an active component.

2. The agent of claim 1, wherein the astaxanthin and/or the ester thereof is derived from a microalga belonging to the genus Haematococcus.

3. A method for increasing adiponectin in blood in a subject in need of an increase in adiponectin in blood, comprising administering to the subject an amount of astaxanthin and/or an ester thereof effective for increasing adiponectin in blood.

4. The method of claim 3, wherein the astaxanthin and/or the ester thereof is derived from a microalga belonging to the genus Haematococcus.
